(19) United States
(12) Patent Application Publication
(10) Pub. No.: US 2025/0114189 A1
(43) Pub. Date: Apr. 10, 2025

(54) PROSTHETIC LEAFLET WITH CUSP EDGE REINFORCING STRUCTURE

(71) Applicant: EDWARDS LIFESCIENCES CORPORATION, Irvine, CA (US)

(72) Inventors: Tamir S. Levi, Zikhron Yaakov (IL); Nikolai Gurovich, Hadera (IL)

(21) Appl. No.: 18/977,753

(22) Filed: Dec. 11, 2024

Related U.S. Application Data
(63) Continuation of application No. PCT/US2023/028325, filed on Jul. 21, 2023.
(60) Provisional application No. 63/369,156, filed on Jul. 22, 2022.

(51) Int. Cl. A61F 2/24

(52) U.S. Cl. CPC A61F 2/2415; A61F 2/2418; A61F 2220/0075; A61F 2240/001

(57) ABSTRACT

A leaflet subassembly for a prosthetic heart valve can include a leaflet having a cusp edge portion and opposite major surfaces. A reinforcing structure can be attached to the leaflet along the cusp edge portion. The reinforcing structure can include a first reinforcing member and a second reinforcing member arranged on the opposite major surfaces of the leaflet. A prosthetic heart valve can include a frame and a leaflet subassembly. The leaflet subassembly can include a leaflet and a reinforcing structure attached to the leaflet along a cusp edge portion of the leaflet. The cusp edge portion can be coupled to the frame via the reinforcing structure. A method of assembling a prosthetic heart valve can include disposing a leaflet at least partially within a frame and attaching the leaflet to the frame by extending a suture through reinforcing members attached to opposite major surfaces of the leaflet.

### CROSS REFERENCE TO RELATED APPLICATION

This application is a continuation of PCT patent application no. PCT/US2023/028325 filed on Jul. 21, 2023, which application claims the benefit of U.S. Provisional Patent Application No. 63/369,156, filed Jul. 22, 2022, each of these applications being incorporated by reference herein in its entirety.

### FIELD

The present disclosure relates to prosthetic heart valves, and to methods and assemblies for forming and installing leaflet assemblies to frames of such prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery device and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic heart valve is mounted, actuating a mechanical actuator that applies an expansion force to the prosthetic heart valve, or by deploying the prosthetic heart valve from a sheath of the delivery device so that the prosthetic heart valve can self-expand to its functional size.

Most expandable prosthetic heart valves include an annular frame or stent and prosthetic leaflets mounted inside the frame. The leaflets may be attached to the frame along the cusp edge portions of the leaflets and at commissure tabs of the leaflets. Mechanically expandable prosthetic heart valves (that is, prosthetic valves that rely on a mechanical actuator for expansion), as well as some balloon-expandable valves and self-expandable valves, elongate axially when radially compressed for delivery into a patient. This can cause overstretching of the leaflets in an axial direction, which can deform and/or damage the leaflets.

### SUMMARY

Described herein are prosthetic heart valves, delivery apparatus, and methods for implanting prosthetic heart valves. The disclosed prosthetic heart valves, delivery apparatus, and methods can, for example, provide enhanced structural integrity of the prosthetic heart valves. In particular, described herein are leaflets having reinforced cusp edge portions that are resistant to tears when sutured to a frame of a prosthetic heart valve or to a skirt attached to the frame of the prosthetic heart valve. As such, the devices and methods disclosed herein can, among other things, overcome one or more of the deficiencies of typical prosthetic heart valves and their delivery apparatus.

A leaflet subassembly for a prosthetic heart valve can comprise a leaflet having a cusp edge portion and a reinforcing structure attached to the leaflet along the cusp edge portion. In addition to these components, a leaflet subassembly can further comprise one or more of the components disclosed herein.

In some examples, a leaflet subassembly can comprise a leaflet having a cusp section, a first major surface, and a second major surface, with the first and second major surface separated by a thickness of the leaflet.

In some examples, a reinforcing structure of a leaflet subassembly can be attached to the leaflet along the cusp edge portion.

In some examples, the reinforcing structure can comprise a first reinforcing member arranged on the first major surface and a second reinforcing member arranged the second major surface.

In some examples, the first reinforcing member and the second reinforcing member can be aligned across the thickness of the leaflet.

In some examples, the reinforcing structure can be attached to the leaflet by a suture extending through both the first reinforcing member and the second reinforcing member and a portion of the leaflet between the first reinforcing member and the second reinforcing member.

In some examples, the first reinforcing member can comprise a plurality of apertures spaced along a length of the first reinforcing member, the second reinforcing member can comprise a plurality of apertures spaced along a length of the second reinforcing member, and the suture can extend through the apertures in the first reinforcing member and the second reinforcing member.

In some examples, the leaflet can comprise a plurality of apertures aligned with the plurality of apertures in the first reinforcing member and the second reinforcing member.

In some examples, the first reinforcing member and the second reinforcing member can track a curvature of the cusp edge portion.

In some examples, a leaflet subassembly for a prosthetic heart valve comprises a leaflet and a reinforcing structure. The leaflet comprises a cusp edge portion, a first major surface, and a second major surface. The first and second major surfaces are separated by a thickness of the leaflet. The reinforcing structure is attached to the leaflet along the cusp edge portion. The reinforcing structure comprising a first reinforcing member arranged on the first major surface and a second reinforcing member arranged the second major surface.

In some examples, a leaflet subassembly comprises one or more of the components recited in Examples 1-8 below.

A prosthetic heart valve can comprise a frame and a leaflet subassembly coupled to the frame. In addition to these components, a prosthetic heart valve can further comprise one or more of the components disclosed herein.

In some examples, the leaflet subassembly can comprise at least one leaflet and a reinforcing structure.

In some examples, the leaflet can comprise a cusp edge portion, a first major surface, and a second major surface such that the first and second major surfaces are separated by a thickness of the leaflet.

In some examples, the reinforcing structure can be attached to the leaflet along the cusp edge portion.

In some examples, the reinforcing structure can comprise a first reinforcing member arranged on the first major surface and a second reinforcing member arranged on the second major surface.

In some examples, the cusp edge portion of the leaflet can be coupled to the frame via the reinforcing structure.

In some examples, the frame can comprise a plurality of struts, and the cusp edge portion can be coupled to at least one strut of the plurality of struts by at least one suture extending through the reinforcing structure.

In some examples, the first reinforcing member can comprise a plurality of apertures spaced along a length of the first reinforcing member, the second reinforcing member can comprise a plurality of apertures spaced along a length of the second reinforcing member, and the at least one suture can extend through one of the apertures in the first reinforcing member and one of the apertures in the second reinforcing member.

In some examples, the at least one suture can form a loop extending around the at least one strut.

In some examples, the prosthetic heart valve can comprise an inner skirt extending along an inner surface of the frame, and the at least one suture can extend through the inner skirt and couples the cusp edge portion to the inner skirt.

In some examples, the prosthetic heart valve can comprise an outer skirt disposed around an outer surface of the frame, and the at least one suture can extend through the outer skirt and couples the inner skirt to the outer skirt.

In some examples, a prosthetic heart valve comprises a frame and a leaflet subassembly. The frame is configured to be radially collapsible and expandable between a radially collapsed configuration and a radially expanded configuration. The leaflet subassembly is situated at least partially within the frame. The leaflet subassembly comprises at least one leaflet and a reinforcing structure. The at least one leaflet comprises a cusp edge portion, a first major surface, and a second major surface. The first and second major surfaces are separated by a thickness of the leaflet. The reinforcing structure is attached to the leaflet along the cusp edge portion. The reinforcing structure comprises a first reinforcing member arranged on the first major surface and a second reinforcing member arranged on the second major surface. The cusp edge portion of the leaflet is coupled to the frame via the reinforcing structure.

In some examples, a prosthetic heart valve comprises one or more of the components recited in Examples 18-39 below.

A method of assembling a prosthetic heart valve can comprise disposing a leaflet at least partially within a frame and attaching the leaflet to the frame. In addition to these steps, a method of assembling a prosthetic heart valve can further comprise one or more of the steps disclosed herein.

In some examples, a method of assembling a prosthetic heart valve can comprise attaching the leaflet to the frame by extending a suture through a first reinforcing member and a second reinforcing member attached to opposite major surfaces of the leaflet along a cusp edge portion of the leaflet, through an inner skirt disposed on an inner surface of the frame, and around a strut of the frame.

In some examples, the suture can extend through the inner skirt at two spaced locations.

In some examples, the suture can extend through an outer skirt disposed around an outer surface of the frame at two spaced locations, and the suture can extend over an outer surface of the outer skirt.

In some examples, the method can comprise forming connected portions between the inner skirt and the outer skirt such that the strut is encapsulated in a space formed between the inner skirt, the outer skirt, and the connected portions.

In some examples, a method of assembling a prosthetic heart valve comprises disposing a leaflet at least partially within a frame and attaching the leaflet to the frame by extending a suture through a first reinforcing member and a second reinforcing member attached to opposite major surfaces of the leaflet along a cusp edge portion of the leaflet, through an inner skirt disposed on an inner surface of the frame, and around a strut of the frame.

In some examples, a method of assembling a prosthetic heart valve comprises one or more of the steps recited in Examples 40-47 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE DRAWINGS

FIG. 1 is a perspective view of a prosthetic heart valve, according to one example.

FIG. 2A is a perspective view of a prosthetic heart valve, according to another example.

FIG. 2B is a perspective view of the prosthetic heart valve of FIG. 2A with the components on the outside of the frame shown in transparent lines.

FIGS. 3A and 3B are plan views of a leaflet subassembly in a laid flat configuration, according to one example.

FIG. 4 is a plan view of a leaflet assembly in a laid flat configuration, according to one example, wherein the leaflet assembly comprises multiple leaflet subassemblies of the type shown in FIGS. 3A-3B.

FIGS. 5A-5C illustrate stitching of a reinforcing structure to a leaflet, according to one example.

FIG. 6A is a side elevation view of a prosthetic heart valve including the leaflet subassemblies of FIGS. 3A and 3B, according to one example.

FIG. 6B is a side elevation view of the prosthetic heart valve of FIG. 6A with the components behind the outer skirt shown for illustrative purposes.

FIG. 6C is an enlarged view of a portion of an inner surface of the frame of FIG. 6A depicting the exemplary leaflet subassemblies.

FIG. 6D is a side elevation view of the prosthetic heart valve similar to FIG. 6B showing exemplary locations of connected portions between an inner skirt of the prosthetic heart valve and the outer skirt.

FIG. 7 is a cross-sectional view of the prosthetic heart valve of FIG. 6B along line 7-7 of FIG. 6B showing an exemplary attachment of the cusp edge portion of the leaflet subassembly to a frame of the prosthetic heart valve via a reinforcing structure of the leaflet subassembly.

FIG. 8 is a cross-sectional view similar to FIG. 7 showing another example of attaching the cusp edge portion of the leaflet assembly to the frame.

FIG. 9 is a cross-sectional view similar to FIG. 7 showing another example of attaching the cusp edge portion of the leaflet assembly to the frame.

FIG. 10 is a side view of an example of a delivery apparatus configured to deliver and implant a radially expandable prosthetic heart valve at an implantation site.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, “e.g.” means “for example,” and “i.e.” means “that is.”

### Introduction to the Disclosure Technology

FIG. 1 shows an exemplary prosthetic valve 100, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the prosthetic heart valves can be implanted within a docking or anchoring device that is implanted within a native heart valve or a vessel. In one example, the prosthetic heart valves can be implanted within a docking device implanted within the pulmonary artery for replacing the function of a diseased pulmonary valve, such as disclosed in U.S. Patent Publication No. 2017/0231756, which is incorporated by reference herein. In another example, the prosthetic heart valves can be implanted within a docking device implanted within or at the native mitral valve, such as disclosed in International Patent Publication No. WO2020/247907, which is incorporated herein by reference. In another example, the prosthetic heart valves can be implanted within a docking device implanted within the superior or inferior vena cava for replacing the function of a diseased tricuspid valve, such as disclosed in U.S. Patent Publication No. 2019/0000615, which is incorporated herein by reference.

In some examples, a leaflet subassembly that can be used in valvular structures for prosthetic heart valves includes a leaflet and a reinforcing structure attached to the leaflet. In some examples, the reinforcing structure includes a pair of reinforcing members attached to opposite sides of the leaflet and adjacent to a cusp edge portion of the leaflet. The reinforcing structure can protect the cusp edge portion from tears when the cusp edge portion is sutured to a frame of a prosthetic heart valve.

### Examples of the Disclosure Technology

FIG. 1 illustrates an exemplary prosthetic heart valve 100, according to one example. The prosthetic heart valve 100 can be radially compressible and expandable between a radially compressed configuration for delivery into a patient and a radially expanded configuration. In the illustrated example, the prosthetic heart valve 100 includes a frame 102 (or stent) and a valvular structure 104 coupled to the frame. The prosthetic heart valve 100 can include an inner skirt 106 arranged on and/or coupled to an inner surface of the frame 102. The prosthetic heart valve 100 can include an outer skirt 108 (or a perivalvular outer sealing member) arranged on and/or coupled to an outer surface of the frame 102. In the illustrated example, the outer skirt 108 is attached to the frame 102 by sutures 130, 132.

The frame 102 can be made of any of various suitable plastically-expandable materials (for example, stainless steel, etc.) or self-expanding materials (for example, Nitinol) as known in the art. When constructed of a plastically-expandable material, the frame 102 (and thus the prosthetic heart valve 100) can be crimped to a radially compressed state on a delivery catheter and then expanded inside a patient by an inflatable balloon or equivalent expansion mechanism. When constructed of a self-expandable material, the frame 102 (and thus the prosthetic heart valve 100) can be crimped to a radially compressed state and restrained in the compressed state by insertion into a sheath or equivalent mechanism of a delivery catheter. Once inside the body, the prosthetic heart valve can be advanced from the delivery sheath, which allows the prosthetic heart valve to expand to its functional size.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

The valvular structure 104 is configured to regulate the flow of blood through the prosthetic heart valve 100, from an inflow end 110 of the prosthetic heart valve 100 to an outflow end 113 of the prosthetic heart valve 100. The valvular structure 104 can include one or more leaflets. In one example, the valvular structure 104 includes three leaflets 114 formed into a leaflet assembly 118, which can be arranged to collapse in a tricuspid arrangement. In some examples, the leaflets 114 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

The leaflets 114 can be secured to one another at their adjacent sides to form commissures 126 of the valvular structure 104. The commissures 126 can be used to couple the valvular structure 104 to the frame 102. For example, the frame 102 can have a plurality of circumferentially spaced slots 124 (or commissure windows) that are adapted to mount the commissures 126 of the valvular structure 104 to the frame. In some examples, the lower edge of the valvular structure 104 can have an undulating, curved scalloped shape and can be secured to the inner skirt 106 by sutures (not shown). The inner skirt 106 can be secured to the frame 102 to couple the lower edge of the valvular structure 104 to the frame.

Additional details regarding the prosthetic heart valve 100 and its various components are described in International Patent Publication No. WO 2018/222799, which is incorporated herein by reference.

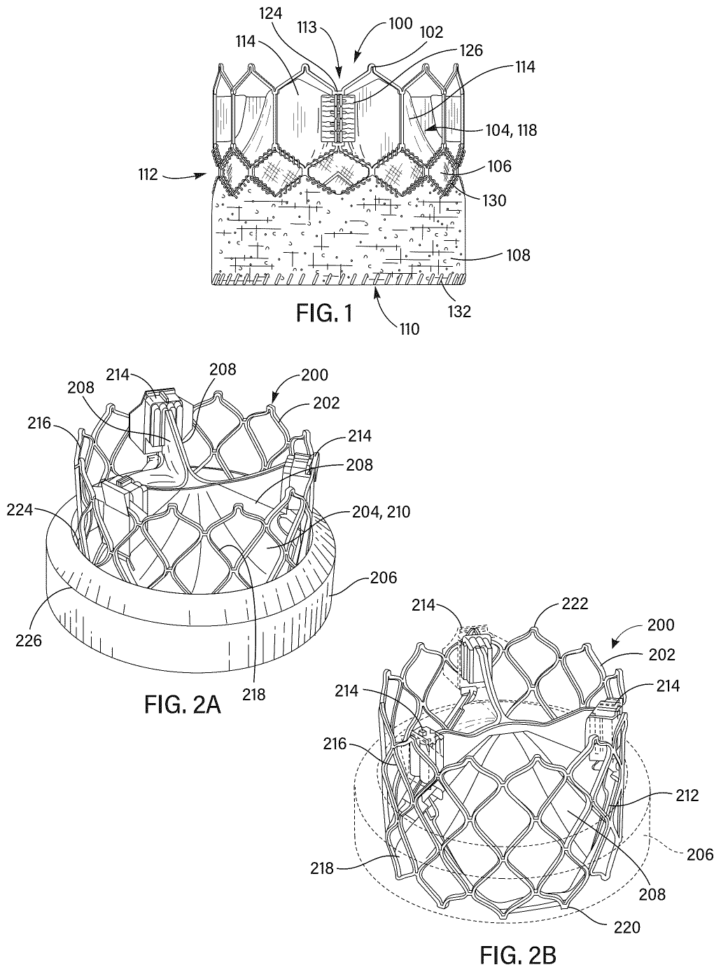

FIGS. 2A and 2B show an exemplary prosthetic heart valve 200, according to another example. In the illustrated example, the prosthetic heart valve 200 includes a frame 202 (or stent), a valvular structure 204, and a sealing member 206. The valvular structure 204 can include three leaflets 208, collectively forming a leaflet assembly 210 that is arranged to collapse in a tricuspid arrangement. Each leaflet 208 can be coupled to the frame 202 along a cusp edge portion 212 of the leaflet (that is, the inflow edge or lower edge of the leaflet in the figure) and at commissures 214 of the valvular structure 204 where adjacent portions of two leaflets 208 are connected to each other. In some examples, a reinforcing element (not shown), such as a fabric strip, can be connected directly to the cusp edge portions of the leaflets 208 and to the struts 216 of the frame 202 to couple the cusp edge portions 212 of the leaflets 208 to the frame 202.

Similar to the frame 102 of FIG. 1, the frame 202 can be made of any of various suitable plastically-expandable materials or self-expanding materials, as known in the art and described above. The frame 202 in the illustrated example of FIGS. 2A and 2B comprises a plurality of circumferentially extending rows of angled struts 216 defining rows of cells 218 (or openings) of the frame 202. The frame 202 can have a cylindrical or substantially cylindrical shape having a constant diameter from an inflow end 220 to an outflow end 222 of the frame as shown, or the frame can vary in diameter along the height of the frame, as disclosed in U.S. Patent Publication No. 2012/0239142, which is incorporated herein by reference.

In the illustrated example, the sealing member 206 is mounted on the outside of the frame 202 and functions to create a seal against the surrounding tissue (for example, the native leaflets and/or native annulus) to prevent or at least minimize paravalvular leakage. The sealing member 206 can have an inner layer 224 and an outer layer 226. In the illustrated example, the inner layer 224 is arranged in contact with the outer surface of the frame 202, while the outer layer 226 is arranged radially outward of the inner layer 224. The sealing member 206 can be connected to the frame 202 using suitable techniques or mechanisms. For example, the scaling member 206 can be sutured to the frame 202 via sutures that can extend around the struts 216 and through the inner layer 224. In other examples, the inner layer 224 can be mounted on the inner surface of the frame 202, while the outer layer 226 is mounted on the outer surface of the frame 202.

In some examples, the outer layer 226 can be configured or shaped to extend radially outward from the inner layer 224 and the frame 202 when the prosthetic heart valve 200 is deployed. When the prosthetic heart valve is fully expanded outside of a patient's body, the outer layer 226 can expand away from the inner layer 224 to create a space between the two layers. Thus, when implanted inside the body, this allows the outer layer 226 to expand into contact with the surrounding tissue, such as a native annulus.

Additional details regarding the prosthetic heart valve 200 and its various components are described in U.S. Patent Publication No. 2018/0028310, which is incorporated herein by reference.

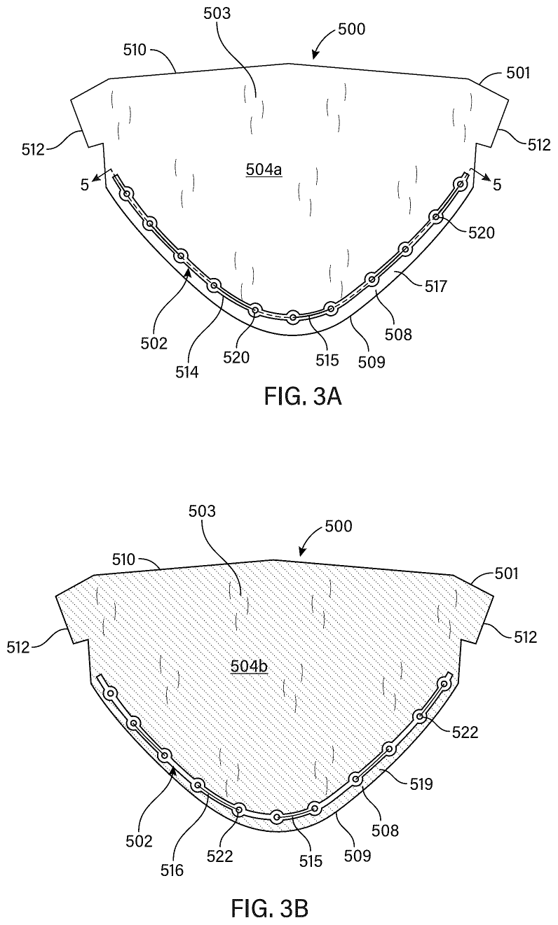

FIGS. 3A and 3B illustrate a leaflet subassembly 500, according to one example. A valvular structure for a prosthetic heart valve can include one or more leaflet subassemblies 500. In the illustrated example, the leaflet subassembly 500 includes a leaflet 501 and a cusp edge reinforcing structure 502 attached to the leaflet 501 to increase a strength of a cusp edge portion (508 in FIGS. 3A and 3B) of the leaflet 501. The cusp edge reinforcing structure 502 can avoid tearing at the cusp edge portion of the leaflet 501 when the cusp edge portion is sutured to a frame of a prosthetic heart valve either directly or via an inner skirt attached to the frame.

The leaflet 501 includes a main body 503 having a first major surface 504a (as depicted in FIG. 3A) and a second major surface 504b (as depicted in FIG. 3B), a cusp edge portion 508 (which can also be referred to as an inflow edge portion of the leaflet or a lower edge portion of the leaflet), a free edge portion 510 (which can also be referred to as an upper edge portion of the leaflet), and a pair of commissure tabs 512 on opposite side edges of the main body 503. The major surfaces 504a, 504b are separated by a thickness of the leaflet 501 (and form opposite major surfaces of the leaflet). The free edge portion 510 can coapt with the free edge portions of adjacent leaflets when the leaflet assembly is in a closed position under pressure from backflow of blood. The commissure tabs 512 of adjacent leaflets can be coupled together to form a leaflet assembly of a valvular structure.

As described for the leaflet 104, the leaflet 501 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

The cusp edge reinforcing structure 502 includes a first reinforcing member 514 and a second reinforcing member 516. The first reinforcing member 514 is arranged on the first major surface 504a along the cusp edge portion 508. The second reinforcing member 516 is arranged on the second major surface 504b along the cusp edge portion 508. The first reinforcing member 514 and the second reinforcing member 516 are attached to the leaflet 501, for example, with a suture 515 (or multiple sutures 515). In some examples, the cusp edge portion 508 can have a curved shape. The attachment of the reinforcing members 514, 516 to the leaflet 501 (for example, via the suture 515) can be such that the reinforcing members 514, 516 track the curvature of the cusp edge portion 508.

In some examples, the position of the first reinforcing member 514 on the first major surface 504a corresponds to the position of the second reinforcing member 516 on the second major surface 504b (that is, the reinforcing members 514, 516 are aligned across the thickness of the leaflet 501) such that the suture 515 can extend through both of the reinforcing members 514, 516 in a direction perpendicular to the major surfaces 504a, 504b. In the illustrated example, the first reinforcing member 514 is offset from a cusp edge 509 by a margin 517. Similarly, the second reinforcing member 516 is offset from a cusp edge 509 by a margin 519. The margins 517, 519 can be selected such that the suture 515 is offset inwardly from the cusp edge 509 (as depicted in FIGS. 3A and 3B).

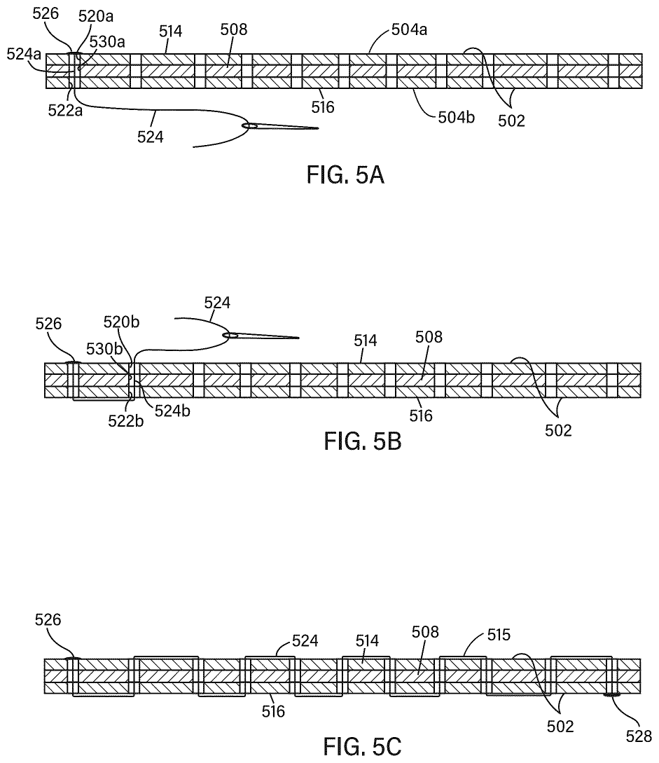

In some examples, as depicted in FIGS. 3A and 3B, apertures 520, 522 (or stitching holes) can be pre-formed in the reinforcing members 514, 516 to receive a needle (or other implement) carrying the suture for forming stitches. The spacing between the apertures 520, 522 on each of the reinforcing members 514, 516 can be even or uneven (for example, it may be desirable to have the apertures closer together where the radius of curvature of the member is smaller and farther apart where the radius of curvature of the member is larger). The leaflet 501 can have pre-formed holes or apertures (for example, 530a, 530b as depicted in FIGS. 5A and 5B) that are aligned with the apertures 520, 522 in the reinforcing members 514, 516. The apertures 520, 522 can be used when attaching the reinforcing members 514, 516 to the leaflet 501 and when attaching the cusp edge portion 508 to a frame or an inner skirt attached to the frame.

The reinforcing members 514, 516 can be in the form of cords or strips. In a particular example, the reinforcing members 514, 516 can be braided sutures, which can be formed of a natural suture material or a synthetic suture material (for example, a polyester suture such as Ethibond suture). In some examples, the suture 515 that attaches the reinforcing members 514, 516 to the leaflet 501 can have a smaller diameter compared to the diameter of either of the reinforcing members 514, 516. In some examples, the suture 515 can be a braided suture, such as an Ethibond suture) having a suture size (gauge) in a range from 3-0 to 5-0, with 4-0 being a specific example, and the reinforcing members 514, 516 can be braided sutures, such as Ethibond sutures, having a suture size (gauge) in a range from 2-0 to 3-0, with 3-0 being a specific example. The suture 515 can be made of any suitable suture material. In another example, the reinforcing members 514, 516 can be braided sutures, while the suture 515 can be a monofilament suture. In other examples, the reinforcing members 514, 516 can comprise a fabric, such as a woven, braided or knitted fabric, such as a PET or UHMW fabric. In further examples, the reinforcing members 514, 516 can comprise natural tissue (for example, pericardial tissue) or layers of non-fabric material, such as made from any of various polymers, such as TPU.

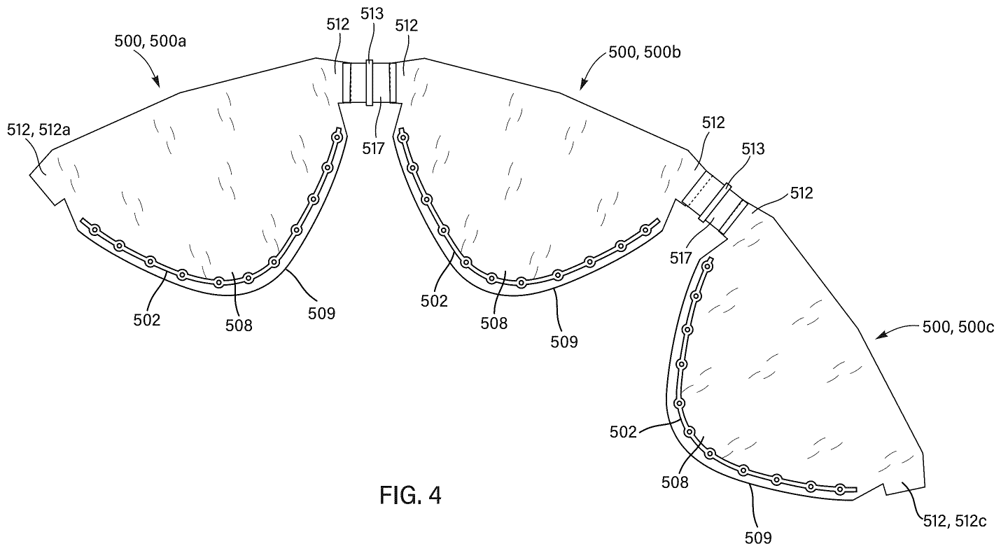

A leaflet assembly can be formed using two or more leaflet subassemblies 500. FIG. 4 shows an example of three leaflet subassemblies 500a, 500b, 500c coupled together to form a leaflet assembly. For example, commissures 513 can be formed between adjacent commissure tabs 512 of adjacent leaflets. In some examples, the commissures 513 can include an attachment member 517 (for example, fabric flexible polymer, or the like) that is attached at opposite ends to the commissure tabs 512. The outer commissure tabs 512a, 512c of the leaflet assembly can be coupled together with another attachment member 517 to form the third commissure 513 and form a leaflet assembly having an annular shape. The commissures 513 formed between the leaflet subassemblies can be used to couple the leaflet assembly to a frame of a prosthetic heart valve.

In some examples, the cusp edge reinforcing structure 502 can be attached to the leaflet 501 prior to attaching the leaflet 501 to other leaflets to form a leaflet assembly. In other examples, the cusp edge reinforcing structure 502 can be attached to the leaflet 501 after attaching the leaflet 501 to other leaflets to form a leaflet assembly.

In some examples, the adjacent ends of first reinforcing members 514 on adjacent leaflets of a leaflet assembly can be connected together to form a single reinforcing member that extends continuously along the first major surfaces of all of the leaflets (or portions of a single reinforcing member extending around a first major surface of the leaflet assembly can provide the first reinforcing members 514 on the first major surfaces of the leaflets of the leaflet assembly). Similarly, in some examples, the adjacent ends of second reinforcing members 516 on adjacent leaflets of a leaflet assembly can be connected together to form a single reinforcing member that extends continuously along the second major surfaces of all of the leaflets (or portions of a single reinforcing member extending around a second major surface of the leaflet assembly can provide the second reinforcing members 516 on the second major surfaces of the leaflets of the leaflet assembly).

FIGS. 5A-5C illustrate attachment of a cusp edge reinforcing structure 502 to a leaflet 501. In this example, an in-and-out stitching pattern (or running stitch pattern) is used, but any stitching pattern that can reliably secure the cusp edge reinforcing structure 502 to the leaflet 501 can be used in other examples.

FIG. 5A shows a portion 524a of a thread 524 (or suture) extending through a first aperture 520a in the first reinforcing member 514, through an aperture 530a in the leaflet 501 (that is, an aperture extending from the first major surface 504a to the second major surface 504b), and through a first aperture 522a in the second reinforcing member 516, completing an “in” portion of a first stitch. FIG. 5B shows another portion 524b of the thread 524 extending through a second aperture 522b in the second reinforcing member 516 that is offset from the first aperture 522a, through an aperture 530b in the leaflet 501 that is aligned with the second aperture 522b, and through a second aperture 520b in the first reinforcing member 514 that is offset from the first aperture 520a and aligned with the apertures 522b, 530b, completing an “out” portion of the first stitch. This sequence of stitching can be repeated at other apertures along the reinforcing members 514, 516 and leaflet 501. FIG. 5C shows the completed suture 515 running along the length of the reinforcing structure 502. The ends of the thread 524 can be knotted or otherwise secured in place (as depicted at 526, 528).

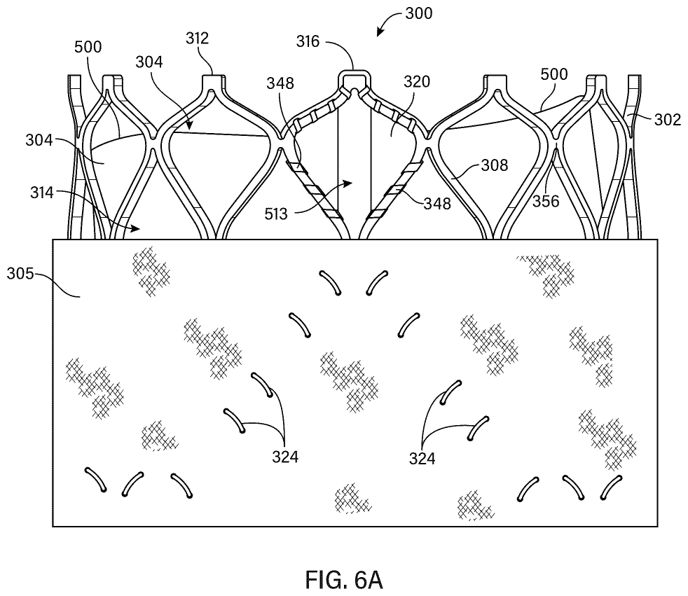

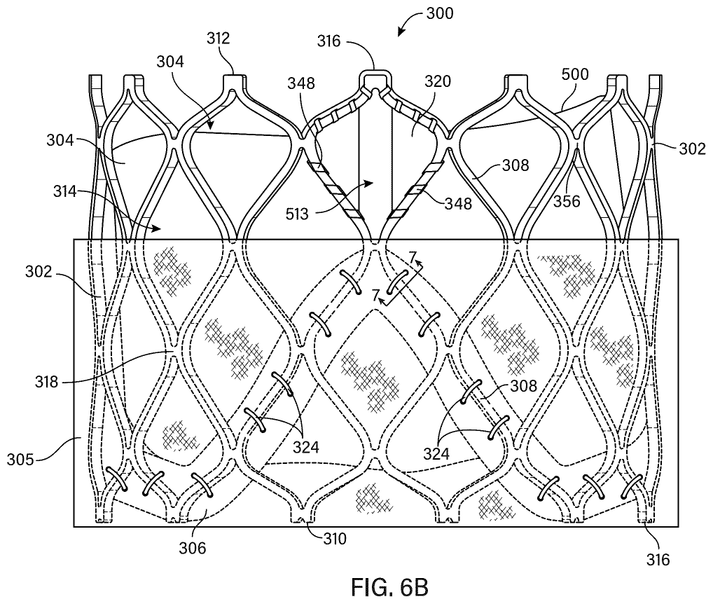

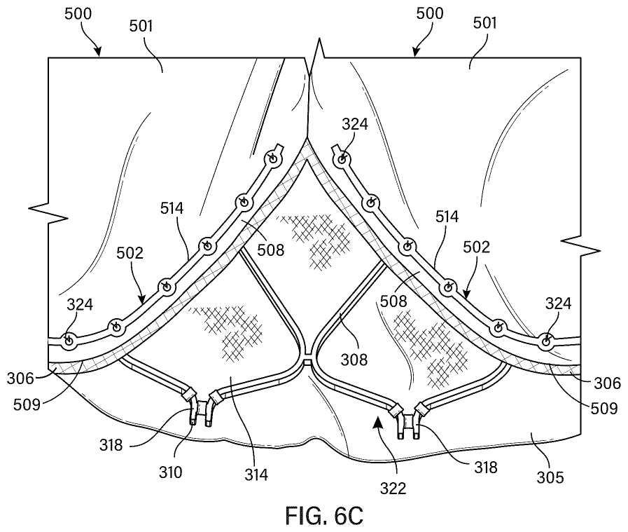

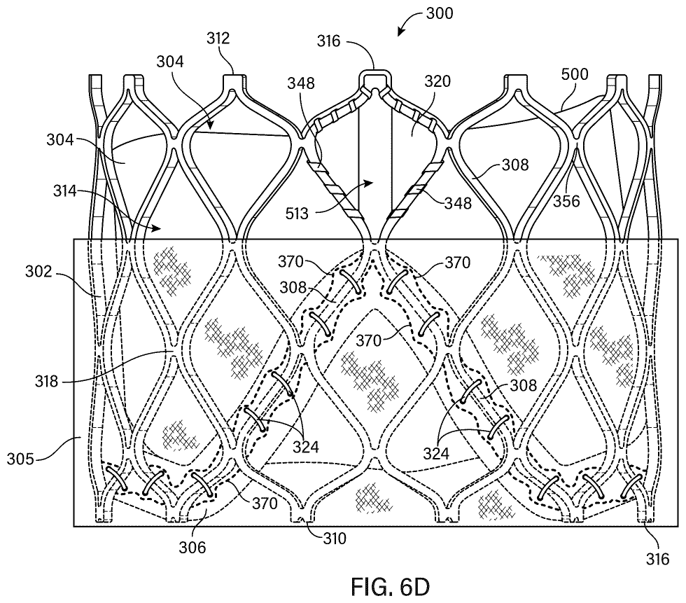

FIGS. 6A-6C illustrate an exemplary prosthetic heart valve 300 that incorporates the leaflet subassembly 500. The prosthetic heart valve 300 includes a frame 302 and a valvular structure 304 arranged within the frame 302. In the illustrated example, the valvular structure 304 comprises a leaflet assembly comprised of three leaflet subassemblies 500 as described herein. The three leaflet subassemblies 500 are arranged to collapse in a tricuspid arrangement. In other examples, the valvular structure 304 can have greater or fewer number of leaflets (for example, one or more of the leaflet subassemblies 500). The leaflet subassemblies 500 are secured to each other via commissures 513 and secured to the frame 302 at the commissures 513.

In the illustrated example, the frame 302 includes an inflow end 310 and an outflow end 312. The frame 302 includes a plurality of angled and interconnected struts 308 arranged into a plurality of circumferentially extending rows of angled struts 308 between the inflow end 310 and the outflow end 312. The rows of struts 308 define rows of cells 314 (or openings) of the frame 302. The struts 308 can intersect at the inflow end 310 and the outflow end 312 to form a plurality of apices 316 that are spaced along the circumference of the frame 302. The struts 308 can further intersect at locations between the inflow end 310 and the outflow end 312 to form strut junctions 318. In the illustrated example, the frame 302 is similar to the frame 202 shown in FIGS. 2A and 2B. In other examples, the frame 302 could have a different structure, such as the structure shown in FIG. 1.

The prosthetic heart valve 300 can include an inner skirt 306 extending along an inner surface of the frame 302 and covering the inner side of at least some of the struts 308. The inner skirt 306 can cover the inner surface of the struts 308 along a scalloped-line pattern (as illustrated in FIG. 6B). There can be one inner skirt 306 extending continuously along the inner surface of the frame 302, or there can be multiple inner skirts 306, where each inner skirt extends along a portion of the frame 302 corresponding to an attachment area for a cusp edge region 508 of one of the leaflets 501. The inner skirt 306 can be made of a suitable tear-resistant material (for example, an implantable polymer such as polyethlene terephthalate (PET), expanded polytetrafluorethylene (ePTFE) or thermoplastic polyurethane (TPU) in the form of a fabric or a non-fabric material).

The prosthetic heart valve 300 can include an outer skirt 305 (or perivalvular outer sealing member) extending around an outer surface of the frame 302 and covering the outer side of at least some of the struts 308. The outer skirt 305 can be made of a suitable tear-resistant material (for example, an implantable polymer such as polyethlene terephthalate (PET), expanded polytetrafluorethylene (ePTFE) or thermoplastic polyurethane (TPU) in the form of a fabric or a non-fabric material).

The inner skirt and/or the outer skirt can be wholly or partly formed of any suitable biological material, synthetic material (for example, any of various polymers), or combinations thereof. In some examples, the inner skirt and/or the outer skirt can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the inner skirt and/or the outer skirt can comprise a fabric without interlaced yarns or fibers or randomly interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the inner skirt and/or the outer skirt can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the inner skirt and/or the outer skirt can comprise a sponge material or foam, such as polyurethane foam. In some examples, the inner skirt and/or the outer skirt can comprise natural tissue, such as pericardium (for example, bovine pericardium, porcine pericardium, equine pericardium, or pericardium from other sources).

In some examples, the cusp edge portions 508 of the leaflets 501 can be attached to the frame 302 via sutures 324 forming stitches that extend through the reinforcing structures 502 of the leaflet subassemblies 500, through one or both of the inner skirt 306 and the outer skirt 305, and around the struts 308. In the illustrated example in FIGS. 6B and 6C, the inner skirt 306 is attached to both the leaflet 501 (via the reinforcing structure 502) and the outer skirt 305. In some examples, the inner skirt 306 can be connected to the outer skirt 305 along undulating struts 308 of the frame 302 (for example, following a scalloped line pattern). In other examples, the sutures 324 can extend through the cusp edge portions of the leaflets (for example, through the reinforcing structures 502), the inner skirt 306, and around selected struts 308 of the frame, but not through the outer skirt 305 (which can be secured to selected struts of the frame 302 with separate sutures).

In some examples, the sutures 324 can be relatively loose and stitching of the outer skirt 305 to the inflow end 310 or the outflow end 312 of the frame 302 (as illustrated for the outer skirt 108 in FIG. 1) can be omitted such that the outer skirt 305 can slide relative to the frame 302 during transition of the frame 302 between the compressed and expanded states without being stretched longitudinally.

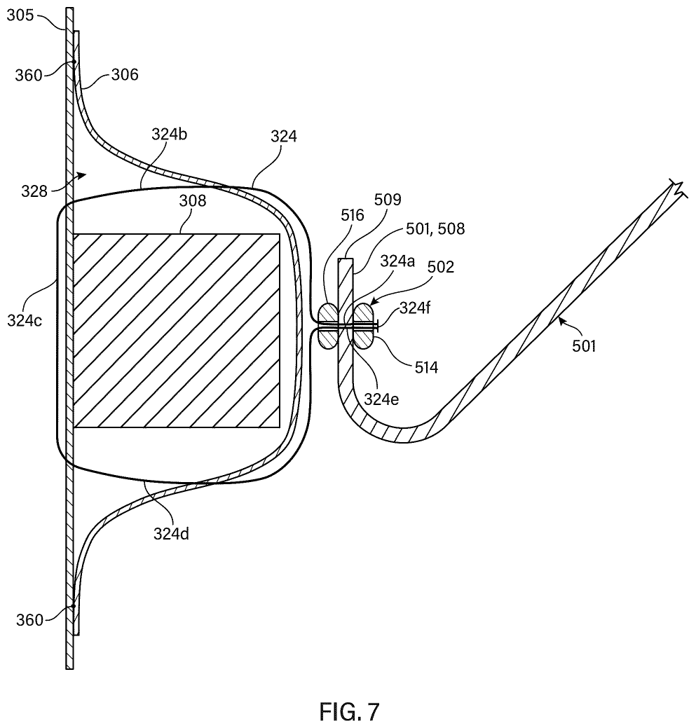

FIG. 7 illustrates a configuration of the suture 324, according to one example. In the example, the suture 324 extends in a radial outward direction through the apertures of the reinforcing structure 502 and the leaflet 501 (as depicted at 324a) and through portions of the inner skirt 306 and the outer skirt 305 above the strut 308 (as depicted at 324b). The suture 324 is looped over an external side of the outer skirt 305 (as depicted at 324c). The suture 324 is extended back through portions of the outer skirt 305 and the inner skirt 306 below the strut 308 (as depicted at 324d) and through the apertures of the reinforcing structure 502 (as depicted at 324c). The ends of the suture 324 can be knotted or otherwise secured together (as depicted at 324f). In this manner, the suture 324 forms a loop around the strut 308. A plurality of spaced sutures 324 looping around respective struts 308 and extending through the skirts 305, 306, the reinforcing structure 502, and the leaflet 501 can be formed (as illustrated in FIGS. 6B and 6C) to couple the cusp edge portions 508 of the leaflet 501 to the frame 302. The sutures 324 can be formed along struts 308 along an undulating path such that the cusp edge regions 508 of the leaflets 501 are coupled to the frame 302 following a scallop line pattern.

In some examples, connected portions 360 are formed between the inner skirt 306 and the outer skirt 305 and around the skirt suture line (formed by sutures 324), which results in encapsulation of the strut 308 in a space 328 between the inner skirt 306 and the outer skirt 305. The connected portions 360 can provide a relatively smooth and gradual transition between the strut 308 and the outer skirt 305 to facilitate proper washout and reduce risks of flow-stagnation areas that could otherwise promote thrombus or pannus formation over time. The connected portions 360 can be formed by bonding or stitching the inner skirt 306 to the outer skirt 305 at locations above sutures 324 (for example, along the paths 370 indicated in FIG. 6D). For example, one or both of the skirts 305, 306 can carry an adhesive layer that can be used to bond the skirts together to form the connected portions. In some examples, the inner skirt 306 can be made of a material that is capable of being bonded or fused to the outer skirt 305 via heat and/or pressure, such as ePTFE or TPU. In some such examples, the inner skirt 306 can be fused to the outer skirt 305 at connected portions 306 without any stitches or separate adhesive layers.

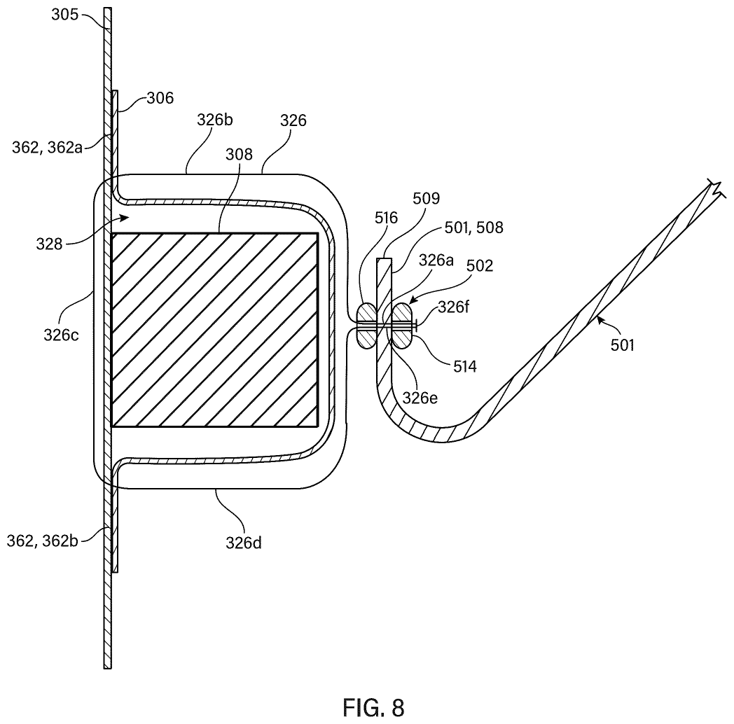

In the illustrated example of FIG. 7, the suture 324 extends through the space 328 and avoids the connected portions 360 (in other words, the suture 324 does not extend through the inner and outer skirts along connected portions 306). FIG. 8 shows an alternative example where a suture 326 can extend through connected portions 362 formed between the inner skirt 306 and the outer skirt 305. In the example of FIG. 8, the suture 326 extends in an outward direction through the apertures of the reinforcing structure 502 and the leaflet 501 (as depicted at 326a) and through connected portion 362a formed between portions of the inner skirt 306 and the outer skirt 305 above the strut 308 (as depicted at 326b). The suture 326 extends over an external side of the outer skirt 305 (as depicted at 326c). The suture 326 extends back through connection portion 362b formed between portions of the outer skirt 305 and the inner skirt 306 below the strut 308 (as depicted at 326d) and through the apertures of the reinforcing structure 502 (as depicted at 326e). The ends of the suture 326 can be knotted or otherwise secured together (as depicted at 326f). The example illustrated in FIG. 8 can extend the durability of the attachment of the leaflet subassembly to the frame by suturing (for example, by reducing the likelihood of enlargement over time of the holes formed in the skirts 305, 306 by passage of the suture 326 through the skirts).

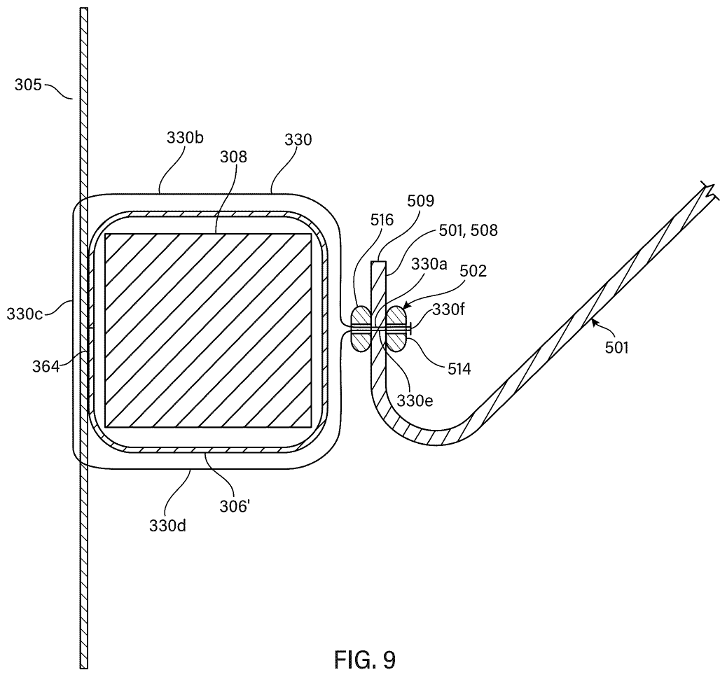

FIG. 9 illustrates another example of attaching the leaflet subassembly 510 to a frame. In the example illustrated in FIG. 9, an inner skirt 306′ is looped around a strut 308 of the frame such that the strut 308 is encapsulated within the inner skirt. A connected portion 364 is formed between the inner skirt 306′ and the outer skirt 305 to attach the inner skirt 306′ to the outer skirt 305. The connected portion 364 can be formed, for example, by bonding (for example, fusing) a portion of the inner skirt 306′ to a portion of the outer skirt 305. The cusp edge portion 508 of the leaflet 501 can be coupled to the frame 302 by a suture 330. In the example, the suture 330 extends in an outward direction through the apertures of the reinforcing structure 502 and the leaflet 501 (as depicted at 330a), above the encapsulated strut 308, and through the outer skirt 305 (as depicted at 330b). The suture 330 extends over an external side of the outer skirt 305 (as depicted at 330c). The suture 330 extends back through the outer skirt 305 (as depicted at 330d) and through the apertures of the reinforcing structure 502 (as depicted at 330c). The ends of the suture 330 can be knotted or otherwise secured together (as depicted at 330f). In the example of FIG. 9, the suture 330 does not penetrate the inner skirt 306′. In other examples, the suture 330 penetrates the inner skirt 306′. A plurality of spaced sutures 330 looping around a strut 308 encapsulated within the inner skirt 306′ and extending through the outer skirt 305, the reinforcing structure 502, and the leaflet 501 can be formed to couple the cusp edge portion 508 of the leaflet 501 to the frame 302.

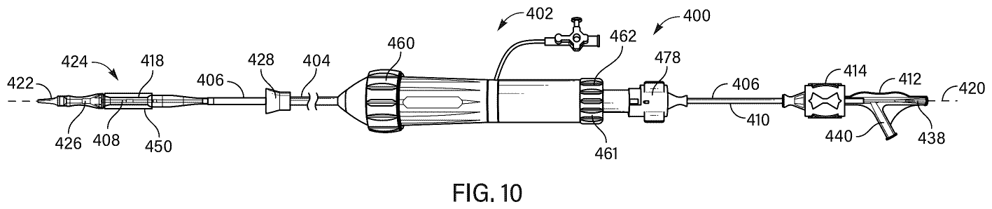

FIG. 10 shows a delivery apparatus 400, according to one example. The delivery apparatus 400 can be used to implant an expandable heart valve (for example, prosthetic heart valve 100 of FIG. 1, prosthetic heart valve 200 of FIGS. 2A-2B, prosthetic heart valve 300 of FIGS. 6A-6D, or any of the other prosthetic heart valves described herein). In some examples, the delivery apparatus 400 is specifically adapted for use in introducing a prosthetic heart valve into a heart.

In the illustrated example, the delivery apparatus 400 is a balloon catheter having a handle 402 and a steerable, outer shaft 404 extending distally from the handle 402. The delivery apparatus 400 can further include an intermediate shaft 406 (which also may be referred to as a balloon shaft) that extends proximally from the handle 402 and distally from the handle 402, the portion extending distally from the handle 402 also extending coaxially through the outer shaft 404. Additionally, the delivery apparatus 400 can further include an inner shaft 408 extending distally from the handle 402 coaxially through the intermediate shaft 406 and the outer shaft 404 and proximally from the handle 402 coaxially through the intermediate shaft 406.

The outer shaft 404 and the intermediate shaft 406 can be configured to translate longitudinally, along a central longitudinal axis 420 of the delivery apparatus 400, relative to one another to facilitate delivery and positioning of a prosthetic heart valve at an implantation site in a patient's body.

The intermediate shaft 406 can include a proximal end portion 410 that extends proximally from a proximal end of the handle 402 to an adaptor 412. A rotatable knob 414 can be mounted on the proximal end portion 410 and can be configured to rotate the intermediate shaft 406 around the central longitudinal axis 420 and relative to the outer shaft 404. The adaptor 412 can include a first port 438 configured to receive a guidewire therethrough and a second port 440 configured to receive fluid (for example, inflation fluid) from a fluid source. The second port 440 can be fluidly coupled to an inner lumen of the intermediate shaft 406.

The intermediate shaft 406 can further include a distal end portion that extends distally beyond a distal end of the outer shaft 404 when a distal end of the outer shaft 404 is positioned away from an inflatable balloon 418 of the delivery apparatus 400. A distal end portion of the inner shaft 408 can extend distally beyond the distal end portion of the intermediate shaft 406. The balloon 418 can be coupled to the distal end portion of the intermediate shaft 406.

In some examples, a distal end of the balloon 418 can be coupled to a distal end of the delivery apparatus 400, such as to a nose cone 422 (as shown in FIG. 10), or to an alternate component at the distal end of the delivery apparatus 400 (for example, a distal shoulder). An intermediate portion of the balloon 418 can overlay a valve mounting portion 424 of a distal end portion of the delivery apparatus 400 and a distal end portion of the balloon 418 can overlay a distal shoulder 426 of the delivery apparatus 400. The valve mounting portion 424 and the intermediate portion of the balloon 418 can be configured to receive a prosthetic heart valve in a radially compressed state. For example, as shown schematically in FIG. 10, a prosthetic heart valve 450 (which can be one of the prosthetic heart valves described herein) can be mounted around the balloon 418, at the valve mounting portion 424 of the delivery apparatus 400.

The balloon shoulder assembly, including the distal shoulder 426, is configured to maintain the prosthetic heart valve 450 (or other medical device) at a fixed position on the balloon 418 during delivery through the patient's vasculature.

The outer shaft 404 can include a distal tip portion 428 mounted on its distal end. The outer shaft 404 and the intermediate shaft 406 can be translated axially relative to one another to position the distal tip portion 428 adjacent to a proximal end of the valve mounting portion 424, when the prosthetic heart valve 450 is mounted in the radially compressed state on the valve mounting portion 424 (as shown in FIG. 4) and during delivery of the prosthetic heart valve to the target implantation site. As such, the distal tip portion 428 can be configured to resist movement of the prosthetic heart valve 450 relative to the balloon 418 proximally, in the axial direction, relative to the balloon 418, when the distal tip portion 428 is arranged adjacent to a proximal side of the valve mounting portion 424.

An annular space can be defined between an outer surface of the inner shaft 408 and an inner surface of the intermediate shaft 406 and can be configured to receive fluid from a fluid source via the second port 440 of the adaptor 412. The annular space can be fluidly coupled to a fluid passageway formed between the outer surface of the distal end portion of the inner shaft 408 and an inner surface of the balloon 418. As such, fluid from the fluid source can flow to the fluid passageway from the annular space to inflate the balloon 418 and radially expand and deploy the prosthetic heart valve 450.

An inner lumen of the inner shaft 408 can be configured to receive a guidewire therethrough for navigating the distal end portion of the delivery apparatus 400 to the target implantation site.

The handle 402 can include a steering mechanism configured to adjust the curvature of the distal end portion of the delivery apparatus 400. In the illustrated example, the handle 402 includes an adjustment member, such as the illustrated rotatable knob 460, which in turn is operatively coupled to the proximal end portion of a pull wire. The pull wire can extend distally from the handle 402 through the outer shaft 404 and has a distal end portion affixed to the outer shaft 404 at or near the distal end of the outer shaft 404. Rotating the knob 460 can increase or decrease the tension in the pull wire, thereby adjusting the curvature of the distal end portion of the delivery apparatus 400. Further details on steering or flex mechanisms for the delivery apparatus can be found in U.S. Pat. No. 9,339,384, which is incorporated by reference herein.

The handle 402 can further include an adjustment mechanism 461 including an adjustment member, such as the illustrated rotatable knob 462, and an associated locking mechanism including another adjustment member, configured as a rotatable knob 478. The adjustment mechanism 461 is configured to adjust the axial position of the intermediate shaft 406 relative to the outer shaft 404 (for example, for fine positioning at the implantation site). Further details on the delivery apparatus 400 can be found in International Publication No. WO 2022/046585, which is incorporated by reference herein.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### ADDITIONAL EXAMPLES

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1: A leaflet subassembly for a prosthetic heart valve comprises a leaflet having a cusp edge portion, a first major surface, and a second major surface, wherein the first and second major surfaces are separated by a thickness of the leaflet; and a reinforcing structure attached to the leaflet along the cusp edge portion, the reinforcing structure comprising a first reinforcing member arranged on the first major surface and a second reinforcing member arranged the second major surface.

Example 2: The leaflet subassembly of any example herein, particularly Example 1, wherein the first reinforcing member and the second reinforcing member are aligned across the thickness of the leaflet.

Example 3: The leaflet subassembly of any example herein, particularly any one of Examples 1-2, wherein the reinforcing structure is attached to the leaflet by a suture extending through both the first reinforcing member and the second reinforcing member and a portion of the leaflet between the first reinforcing member and the second reinforcing member.

Example 4: The leaflet subassembly of any example herein, particularly Example 3, wherein the first reinforcing member comprises a plurality of apertures spaced along a length of the first reinforcing member, and wherein the second reinforcing member comprises a plurality of apertures spaced along a length of the second reinforcing member.

Example 5: The leaflet subassembly of any example herein, particularly Example 4, wherein the suture extends through the apertures in the first reinforcing member and the second reinforcing member.

Example 6: The leaflet subassembly of any example herein, particularly any one of Examples 4-5, wherein the leaflet comprises a plurality of apertures aligned with the plurality of apertures in the first reinforcing member and the second reinforcing member.

Example 7: The leaflet subassembly of any example herein, particularly any one of Examples 1-6, wherein the first reinforcing member and the second reinforcing member track a curvature of the cusp edge portion.

Example 8: The leaflet subassembly of any example herein, particularly any one of Examples 1-7, wherein each of the first reinforcing member and second reinforcing member comprises a braided suture.

Example 9: A leaflet assembly for a prosthetic heart valve comprises a plurality of leaflet subassemblies, each of the leaflet subassemblies comprises a leaflet having a cusp edge portion, a first major surface, and a second major surface, wherein the first and second major surfaces are separated by a thickness of the leaflet, wherein the leaflet assembly comprises a reinforcing structure attached to the leaflet along the cusp edge portion, the reinforcing structure comprising a first reinforcing member arranged on the first major surface and a second reinforcing member arranged on the second major surface, and wherein the leaflet assembly comprises a plurality of commissures connecting the leaflet subassemblies together.

Example 10: The leaflet assembly of any example herein, particularly Example 9, wherein the first reinforcing member and the second reinforcing member of each leaflet subassembly are aligned across the thickness of the leaflet of the leaflet subassembly.

Example 11: The leaflet assembly of any example herein, particularly any one of Examples 9-10, wherein the reinforcing structure of each leaflet subassembly is attached to the leaflet of each leaflet subassembly by a suture extending through both the first reinforcing member and the second reinforcing member of the reinforcing structure and a portion of the leaflet between the first reinforcing member and the second reinforcing member of the reinforcing structure.

Example 12: The leaflet assembly of any example herein, particularly Example 11, wherein the first reinforcing member of each leaflet subassembly comprises a plurality of apertures spaced along a length of the first reinforcing member, and wherein the second reinforcing member of each leaflet subassembly comprises a plurality of apertures spaced along a length of the second reinforcing member.

Example 13: The leaflet assembly of any example herein, particularly Example 12, wherein the suture of each leaflet subassembly extends through the apertures in the first reinforcing member and the second reinforcing member of the leaflet subassembly.

Example 14: The leaflet assembly of any example herein, particularly any one of Examples 12-13, wherein the leaflet of each leaflet subassembly comprises a plurality of apertures aligned with the plurality of apertures in the first reinforcing member and the second reinforcing member of the leaflet subassembly.

Example 15: The leaflet assembly of any example herein, particularly any one of Examples 9-14, wherein the reinforcing structure of each leaflet subassembly tracks a curvature of the cusp edge portion of each leaflet subassembly.

Example 16: The leaflet assembly of any example herein, particularly any one of Examples 9-15, wherein each of the first reinforcing member and second reinforcing member of each leaflet subassembly comprises a braided suture.

Example 17: The leaflet assembly of any example herein, particularly any one of Examples 9-16, wherein each leaflet subassembly comprises a pair of commissure tabs located on opposite sides of the leaflet of the leaflet subassembly, and wherein each commissure is formed between adjacent commissure tabs of adjacent leaflet subassemblies.

Example 18: A prosthetic heart valve comprises a frame configured to be radially collapsible and expandable between a radially collapsed configuration and a radially expanded configuration; and a leaflet subassembly situated at least partially within the frame. The leaflet subassembly comprises at least one leaflet having a cusp edge portion, a first major surface, and a second major surface, wherein the first and second major surfaces are separated by a thickness of the leaflet; and a reinforcing structure attached to the leaflet along the cusp edge portion, the reinforcing structure comprising a first reinforcing member arranged on the first major surface and a second reinforcing member arranged on the second major surface, wherein the cusp edge portion of the leaflet is coupled to the frame via the reinforcing structure.

Example 19: The prosthetic heart valve of any example herein, particularly Example 18, wherein the frame comprises a plurality of struts.

Example 20: The prosthetic heart valve of any example herein, particularly Example 19, wherein the cusp edge portion is coupled to at least one strut of the plurality of struts by at least one suture extending through the reinforcing structure.

Example 21: The prosthetic heart valve of any example herein, particularly Example 20, wherein the first reinforcing member comprises a plurality of apertures spaced along a length of the first reinforcing member, wherein the second reinforcing member comprises a plurality of apertures spaced along a length of the second reinforcing member, and wherein the at least one suture extends through one of the apertures in the first reinforcing member and one of the apertures in the second reinforcing member.

Example 22: The prosthetic heart valve of any example herein, particularly any one of Examples 20-21, wherein the at least one suture forms a loop extending around the at least one strut.

Example 23: The prosthetic heart valve of any example herein, particularly any one of Examples 20-22, further comprises an inner skirt extending along an inner surface of the frame, wherein the at least one suture extends through the inner skirt and couples the cusp edge portion to the inner skirt.

Example 24: The prosthetic heart valve of any example herein, particularly Example 23, wherein the at least one suture extends through the inner skirt on opposite sides of the at least one strut.

Example 25: The prosthetic heart valve of any example herein, particularly any one of Examples 23-24, further comprises an outer skirt disposed around an outer surface of the frame, wherein the at least one suture extends through the outer skirt and couples the inner skirt to the outer skirt.

Example 26: The prosthetic heart valve of any example herein, particularly Example 25, wherein the at least one suture extends through the outer skirt on opposite sides of the at least one strut and extends over a portion of an external side of the outer skirt.

Example 27: The prosthetic heart valve of any example herein, particularly any one of Examples 25-26, wherein connected portions are formed between the inner skirt and the outer skirt on opposite sides of the at least one strut, and wherein the at least one strut is encapsulated in a space between the inner skirt, the outer skirt, and the connected portions.

Example 28: The prosthetic heart valve of any example herein, particularly Example 27, wherein the connected portions comprise portions of the outer skirt and the inner skirt bonded together.

Example 29: The prosthetic heart valve of any example herein, particularly Example 27, wherein the connected portions comprise portions of the outer skirt and the inner skirt stitched together.

Example 30: The prosthetic heart valve of any example herein, particularly any one of Examples 27-29, wherein the at least one suture extends through the space between the inner skirt and the outer skirt.

Example 31: The prosthetic heart valve of any example herein, particularly any one of Examples 27-29, wherein the at least one suture extends through the connected portions.

Example 32: The prosthetic heart valve of any example herein, particularly any one of Examples 20-22, further comprises an inner skirt encapsulating the at least one strut.

Example 33: The prosthetic heart valve of any example herein, particularly Example 32, further comprises an outer skirt disposed around an outer surface of the frame and a connected portion formed between the outer skirt and the inner skirt.

Example 34: The prosthetic heart valve of any example herein, particularly Example 33, wherein the at least one suture extends through the outer skirt and couples the cusp edge portion to the outer skirt.

Example 35: The prosthetic heart valve of any example herein, particularly any one of Examples 19-34, wherein the first reinforcing member and the second reinforcing member track a curvature of the cusp edge portion.

Example 36: The prosthetic heart valve of any example herein, particularly any one of Examples 19-35, wherein each of the first reinforcing member and second reinforcing member comprises a braided suture.

Example 37: The prosthetic heart valve of any example herein, particularly Example 19, further comprises an inner skirt extending along an inner surface of the frame, wherein the leaflet is sutured to the inner skirt via the reinforcing structure and along at least one strut of the plurality of struts.

Example 38: The prosthetic heart valve of any example herein, particularly Example 37, further comprises an outer skirt disposed around an outer surface of the frame, wherein the inner skirt is sutured to the outer skirt along the at least one strut.

Example 39: The prosthetic heart valve of any example herein, particularly any one of Examples 18-38, wherein the leaflet assembly comprises a plurality of leaflets.

Example 40: A method of assembling a prosthetic heart valve comprises disposing a leaflet at least partially within a frame; and attaching the leaflet to the frame by extending a suture through a first reinforcing member and a second reinforcing member attached to opposite major surfaces of the leaflet along a cusp edge portion of the leaflet, through an inner skirt disposed on an inner surface of the frame, and around a strut of the frame.

Example 41: The method of any example herein, particularly Example 40, wherein the suture extends through the inner skirt at two spaced locations.

Example 42: The method of any example herein, particularly any one of Examples 40-41, wherein the suture extends through an outer skirt disposed around an outer surface of the frame.

Example 43: The method of any example herein, particularly Example 42, wherein the suture extends through the outer skirt at two spaced locations.

Example 44: The method of any example herein, particularly Example 42, wherein the suture extends over an outer surface of the outer skirt.

Example 45: The method of any example herein, particularly Example 42, further comprises forming connected portions between the inner skirt and the outer skirt, wherein the strut is encapsulated in a space formed between the inner skirt, the outer skirt, and the connected portions.

Example 46: The method of any example herein, particularly Example 45, wherein the suture extends through the space.

Example 47: The method of any example herein, particularly Example 45, wherein the suture extends through the connected portions.

Example 48: A delivery assembly comprises a delivery apparatus; and a prosthetic heart valve coupled to the delivery apparatus. The prosthetic heart valve comprises a frame that is radially collapsible and expandable between a collapsed configuration and an expanded configuration; and at least one leaflet having a cusp edge portion and opposite major surfaces; and a reinforcing structure attached to the leaflet along the cusp edge portion, the reinforcing structure comprising a first reinforcing member and a second reinforcing member arranged on the opposite major surfaces of the leaflet, wherein the cusp edge portion of the leaflet is coupled to the frame via the reinforcing structure.

Example 49: The delivery assembly of any example herein, particularly Example 48, wherein the frame comprises a plurality of struts.

Example 50: The delivery assembly of any example herein, particularly Example 49, wherein the cusp edge portion is coupled to at least one strut of the plurality of struts by at least one suture extending through the reinforcing structure and the leaflet.

Example 51: The delivery assembly of any example herein, particularly any one of Examples 48-50, further comprises an inner skirt disposed around an inner surface of the frame, wherein the cusp edge portion of the leaflet is coupled to the frame via the reinforcing structure and the inner skirt.

Example 52: The delivery assembly of any example herein, particularly Example 49, further comprises an outer skirt disposed around an outer surface of the frame, wherein the inner skirt is attached to the outer skirt such that the at least one strut is encapsulated between the inner skirt and the outer skirt.

Example 53: The delivery assembly of any example herein, particularly any one of Examples 48-52, wherein the first reinforcing member and the second reinforcing member track a curvature of the cusp edge portion.

Example 54: A delivery assembly comprises a delivery apparatus; and a prosthetic heart valve coupled to the delivery apparatus. The prosthetic heart valve comprises a frame that is radially collapsible and expandable between a collapsed configuration and an expanded configuration; and a plurality of leaflet subassemblies coupled to the frame. Each of the leaflet subassemblies comprises a leaflet having a cusp edge portion and opposite major surfaces; and a reinforcing structure attached to the leaflet along the cusp edge portion, the reinforcing structure comprising a first reinforcing member and a second reinforcing member arranged on the opposite major surfaces of the leaflet, wherein the cusp edge portion of the leaflet is coupled to the frame via the reinforcing structure.

Example 55: The leaflet subassembly, leaflet assembly, prosthetic heart valve, or delivery assembly of any example herein, particularly any one of Examples 1-39 and Examples 48-54, wherein the leaflet subassembly, leaflet assembly, prosthetic heart valve, or delivery assembly is sterilized.

Example 56: A method comprising sterilizing the leaflet subassembly, leaflet assembly, prosthetic heart valve, or delivery assembly of any example herein, particularly any one of Examples 1-39 and Examples 48-54.

The features described herein with regard to any example can be combined with other features described in any one or more of the other examples, unless otherwise stated. For example, any one or more of the features of one leaflet assembly can be combined with any one or more features of another leaflet assembly. As another example, any one or more features of one prosthetic heart valve can be combined with any one or more features of another prosthetic heart valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A leaflet subassembly for a prosthetic heart valve, the leaflet subassembly comprising:
a leaflet having a cusp edge portion, a first major surface, and a second major surface, wherein the first and second major surfaces are separated by a thickness of the leaflet; and
a reinforcing structure attached to the leaflet along the cusp edge portion, the reinforcing structure comprising a first reinforcing member arranged on the first major surface and a second reinforcing member arranged the second major surface.

2. The leaflet subassembly of claim 1, wherein the first reinforcing member and the second reinforcing member are aligned across the thickness of the leaflet.

3. The leaflet subassembly of claim 1, wherein the reinforcing structure is attached to the leaflet by a suture extending through both the first reinforcing member and the second reinforcing member and a portion of the leaflet between the first reinforcing member and the second reinforcing member.

4. The leaflet subassembly of claim 3, wherein the first reinforcing member comprises a plurality of apertures spaced along a length of the first reinforcing member, wherein the second reinforcing member comprises a plurality of apertures spaced along a length of the second reinforcing member, and wherein the suture extends through the apertures in the first reinforcing member and the second reinforcing member.

5. The leaflet subassembly of claim 4, wherein the leaflet comprises a plurality of apertures aligned with the plurality of apertures in the first reinforcing member and the second reinforcing member.

6. The leaflet subassembly of claim 1, wherein the first reinforcing member and the second reinforcing member track a curvature of the cusp edge portion.

7. A prosthetic heart valve comprising:
a frame configured to be radially collapsible and expandable between a radially collapsed configuration and a radially expanded configuration; and
a leaflet subassembly situated at least partially within the frame, the leaflet subassembly comprising:
at least one leaflet having a cusp edge portion, a first major surface, and a second major surface, wherein the first and second major surfaces are separated by a thickness of the leaflet; and
a reinforcing structure attached to the leaflet along the cusp edge portion, the reinforcing structure comprising a first reinforcing member arranged on the first major surface and a second reinforcing member arranged on the second major surface, wherein the cusp edge portion of the leaflet is coupled to the frame via the reinforcing structure.

8. The prosthetic heart valve of claim 7, wherein the frame comprises a plurality of struts, and wherein the cusp edge portion is coupled to at least one strut of the plurality of struts by at least one suture extending through the reinforcing structure.

9. The prosthetic heart valve of claim 8, wherein the first reinforcing member comprises a plurality of apertures spaced along a length of the first reinforcing member, wherein the second reinforcing member comprises a plurality of apertures spaced along a length of the second reinforcing member, and wherein the at least one suture extends through one of the apertures in the first reinforcing member and one of the apertures in the second reinforcing member.

10. The prosthetic heart valve of claim 8, wherein the at least one suture forms a loop extending around the at least one strut.

11. The prosthetic heart valve of claim 8, further comprising an inner skirt extending along an inner surface of the frame, wherein the at least one suture extends through the inner skirt and couples the cusp edge portion to the inner skirt.

12. The prosthetic heart valve of claim 11, further comprising an outer skirt disposed around an outer surface of the frame, wherein the at least one suture extends through the outer skirt and couples the inner skirt to the outer skirt.

13. The prosthetic heart valve of claim 12, wherein connected portions are formed between the inner skirt and the outer skirt on opposite sides of the at least one strut, and wherein the at least one strut is encapsulated in a space between the inner skirt, the outer skirt, and the connected portions.

14. The prosthetic heart valve of claim 13, wherein the connected portions comprise portions of the outer skirt and the inner skirt that are one or both of bonded together and stitched together.

15. The prosthetic heart valve of claim 13, wherein the at least one suture extends through the space between the inner skirt and the outer skirt.

16. The prosthetic heart valve of claim 13, wherein the at least one suture extends through the connected portions.

17. The prosthetic heart valve of claim 8, further comprising:
an inner skirt encapsulating the at least one strut;
an outer skirt disposed around an outer surface of the frame; and
a connected portion formed between the outer skirt and the inner skirt.

18. The prosthetic heart valve of claim 17, wherein the at least one suture extends through the outer skirt and couples the cusp edge portion to the outer skirt.

19. The prosthetic heart valve of claim 8, further comprising an inner skirt extending along an inner surface of the frame and an outer skirt disposed around an outer surface of the frame, wherein the leaflet is sutured to the inner skirt via the reinforcing structure and along at least one strut of the plurality of struts, and wherein the inner skirt is sutured to the outer skirt along the at least one strut.

20. A method of assembling a prosthetic heart valve, the method comprising:
disposing a leaflet at least partially within a frame; and
attaching the leaflet to the frame by extending a suture through a first reinforcing member and a second reinforcing member attached to opposite major surfaces of the leaflet along a cusp edge portion of the leaflet, through an inner skirt disposed on an inner surface of the frame, and around a strut of the frame.
